(19) United States
(12) Patent Application Publication
(10) Pub. No.: US 2025/0246293 A1
(43) Pub. Date: Jul. 31, 2025

(54) HEALTHCARE SERVICE ESTIMATING SYSTEM AND METHOD OF USE

(71) Applicant: Nagapradeep Nagajothi, Massillon, OH (US)

(72) Inventor: Nagapradeep Nagajothi, Massillon, OH (US)

(21) Appl. No.: 19/036,618

(22) Filed: Jan. 24, 2025

Related U.S. Application Data
(60) Provisional application No. 63/625,365, filed on Jan. 26, 2024.

(51) Int. Cl. G16H 40/20; G06Q 30/0283

(52) U.S. Cl. CPC G16H 40/20; G06Q 30/0283

(57) ABSTRACT

The present invention is a healthcare service estimating system including a patient application, enabling users to input healthcare service requirements, search for providers, request quotes, and review estimates. A provider application enables healthcare providers to manage profiles, respond to quote inquiries, and configure automated responses for frequently requested services. A server system, communicatively coupled to the applications, incorporates a patient database, a provider database, an automated quote module for generating good faith estimates, and an application server for managing data and interactions. The system is adapted to adjust estimates based on insurance coverage. The system offers real-time cost transparency, regulatory compliance, and empowers patients to make informed decisions and promotes efficient provider interactions.

### CROSS-REFERENCE TO RELATED APPLICATION

The present application claims priority to, and the benefit of, U.S. Provisional Application No. 63/625,365 which was filed on Jan. 26, 2024 and is incorporated herein by reference in its entirety

### FIELD OF THE INVENTION

The present invention generally relates to healthcare cost transparency systems. More specifically, the invention relates to a comprehensive healthcare service estimating system designed to enable patients to obtain and compare cost estimates for medical services before committing to treatment. The system comprises a patient-facing application for inputting service requirements, searching for healthcare providers, and requesting quotes, and a provider-facing application for managing profiles and responding to patient inquiries. The system further includes a server system hosting a patient database, a provider database, and an automated quote module configured to generate good faith estimates based on predefined rules and provider inputs. Accordingly, the present disclosure makes specific reference thereto. Nonetheless, it is to be appreciated that aspects of the present invention are also equally applicable to other like applications, devices, and methods of manufacture.

### BACKGROUND

By way of background, many individuals require healthcare services but often hesitate to seek necessary treatments due to uncertainty and anxiety surrounding the potential expenses. A lot of times, patients do not have transparency regarding the costs of medical services. For patients, the process of obtaining accurate cost estimates is often cumbersome, involving direct communication with multiple providers, unclear pricing information, and a lack of standardized methods for comparing costs. Even when healthcare providers offer estimates, the estimates may lack consistency or fail to account for factors such as insurance coverage, location, or service complexity. As a result, patients frequently face unexpected bills that exacerbate financial stress.

Healthcare providers, on the other hand, struggle with managing pricing inquiries efficiently. Responding to patient requests manually can be time-consuming, particularly for high-demand or frequently requested services. Currently, there are no systems that bridge the gap between patients and providers. Individuals desire a system that provides accurate, real-time cost transparency between patients and providers.

Therefore, there exists a long-felt need in the art for a healthcare service estimating system that improves transparency in healthcare costs and enables patients to make informed decisions regarding medical services. There is a long-felt need for a system that enables patients to easily compare costs from multiple healthcare providers and access accurate, good faith estimates. Additionally, there is a long-felt need in the art for a healthcare service estimating software. Furthermore, there exists a need for a system that automates provider responses to quote inquiries. More specifically, there exists a long-felt need in the art for a healthcare service estimating software that enables a user to obtain a quote for the cost of healthcare services from a healthcare provider. Finally, there is a long-felt need for a comprehensive platform that ensures compliance with healthcare regulations, improves trust between patients and providers, and encourages the timely utilization of healthcare services.

The subject matter disclosed and claimed herein, in one embodiment, comprises a healthcare service estimating system designed to provide patients with cost estimates for healthcare services from multiple providers. The system includes a patient application installed on an electronic computing device, such as a smartphone, enabling patients to log in, input healthcare service requirements, search for relevant providers, request quotes, and review cost estimates. The system further includes a provider application installed on provider devices, enabling healthcare providers to manage profiles, review patient requests, and submit manual or automated responses to inquiries. A server system is communicatively coupled to the applications via a communication network. The server system includes a patient database for storing patient profiles and past quote requests, a provider database for maintaining provider details and pre-configured responses, and an automated quote module that generates good faith estimates based on predefined rules and provider inputs.

In this manner, the healthcare service estimating system of the present invention accomplishes all of the foregoing objectives and provides an innovative solution to cost transparency challenges in healthcare. The system empowers patients to compare provider options and make informed decisions by offering real-time, easily accessible cost estimates. The system enhances efficiency and response time by enabling providers to set up automatic quote responses for frequently requested services.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a healthcare service estimating system. The system includes a patient application installed on an electronic computing device, a provider application installed on a provider computing device, and a server system communicatively coupled to the patient application and the provider application via a communication network.

In another aspect, the patient application is configured to enable patients to log in using credentials, enable patients to input healthcare service requirements, display relevant healthcare service providers based on patient criteria including location, urgency, and budget, and facilitate submission of quote requests to selected healthcare service providers and review of received quotes.

In another embodiment, the provider application is configured to enable healthcare service providers to log in using credentials, display patient requests for healthcare service quotes, enable providers to submit manual or automated responses to quote requests, and facilitate management of provider profiles and response configurations for frequently requested services.

In one embodiment, the server system includes a patient database configured to store patient profiles, healthcare service preferences, and past requests, a provider database configured to store provider profiles, pricing details, and pre-configured response settings, an automated quote module configured to generate good faith estimates based on patient requests and provider inputs, and an application server hosting backend application logic to process requests and manage database operations.

In still another aspect, a method for providing healthcare service estimates to patients is described. The method includes the steps of logging in a patient to a healthcare service estimating system via a patient application using credentials, enabling the patient to input one or more healthcare service requirements, displaying a list of healthcare service providers relevant to the patient's criteria, the criteria including location, urgency, and service type, receiving the selection of one or more providers by the patient and transmitting quote requests to the selected providers, generating good faith estimates for the requested services and/or combinations of multiple procedures for the requested services, by the providers and displaying the estimates to the patient for comparison and selection, and facilitating the patient's selection of a healthcare service provider and processing payment for the selected service via a secure payment module.

In yet another embodiment, a software patient application for estimating healthcare service costs is disclosed. The application is configured to enable patient login and profile management, facilitate input of healthcare service requirements, display healthcare service providers based on patient criteria, transmit quote requests to selected providers, and display received quotes for patient review and selection.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a schematic view of healthcare service estimating system of the present invention in accordance with the disclosed architecture;

FIG. 2 illustrates a flow chart depicting a process of providing healthcare service quotes to patients in accordance with one embodiment of the present invention;

FIG. 3 illustrates a flow chart depicting a provider-side process for providing estimates of healthcare services in accordance with one embodiment of the present invention;

FIG. 4 illustrates a flow chart depicting a process of generating estimates for healthcare services considering insurance of the patients in accordance with the disclosed architecture;

FIG. 5 illustrates an exemplary user interface displayed by the patient application for requesting service providers in accordance with one embodiment of the present invention;

FIG. 6 illustrates another user interface displayed by the patient application showing a list of relevant healthcare services providers in accordance with the disclosed architecture; and

FIG. 7 illustrates an exemplary user interface for displaying information of a provider which enables a patient to make an informed decision in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a healthcare service estimating system that improves transparency in healthcare costs and enables patients to make informed decisions regarding medical services. There is a long-felt need for a system that enables patients to easily compare costs from multiple healthcare providers and access accurate, good faith estimates. Additionally, there is a long-felt need in the art for a healthcare service estimating software. Furthermore, there exists a need for a system that automates provider responses to quote inquiries. More specifically, there exists a long-felt need in the art for a healthcare service estimating software that enables a user to obtain a quote for the cost of healthcare services from a healthcare provider. Finally, there is a long-felt need for a comprehensive platform that ensures compliance with healthcare regulations, improves trust between patients and providers, and encourages the timely utilization of healthcare services.

The present invention, in one exemplary embodiment, is a method for providing healthcare service estimates to patients. The method includes the steps of logging in a patient to a healthcare service estimating system via a patient application using credentials, enabling the patient to input one or more healthcare service requirements, displaying a list of healthcare service providers relevant to the patient's criteria, the criteria including location, urgency, and service type, receiving the selection of one or more providers by the patient and transmitting quote requests to the selected providers, generating good faith estimates for the requested services and/or combinations of multiple procedures for the requested services, by the providers and displaying the estimates to the patient for comparison and selection, and facilitating the patient's selection of a healthcare service provider and processing payment for the selected service via a secure payment module. The system may be accessed through an application on any smart device or via an internet website

Reference will now be made in detail to the present preferred embodiments of the invention, examples of which are illustrated in the accompanying drawings. Wherever possible, the same reference numerals are used in the drawings and the description to refer to the same or like parts.

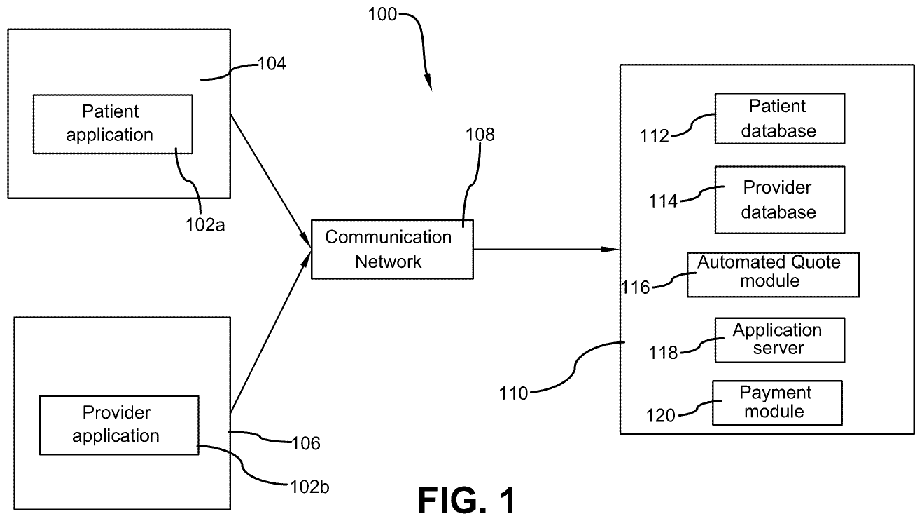

Referring initially to the drawings, FIG. 1 illustrates a schematic view of healthcare service estimating system of the present invention in accordance with the disclosed architecture. The healthcare service estimating system 100 of the present invention is configured to improve transparency in healthcare costs by enabling patients to receive estimates for healthcare services before committing to treatment. The system 100 enables patients to receive healthcare services' costs from different healthcare service providers and compare costs and make informed choices. More specifically, the system 100 includes a software patient application 102a configured to be used by patients for accessing functionalities offered by the system 100. The application 102a can be installed in an electronic computing device 104 such as a smartphone. The application 102a provides various user interfaces for enabling a user to input healthcare services requirements and select a healthcare service provider.

The system 100 includes a healthcare service provider application 102b installed in a provider device 106. The healthcare service provider application 102b enables healthcare providers to manage their profiles, view patient requests, and respond to quote inquiries of patients. It should be noted that the system 100 provides cost transparency for patients seeking medical services. Using the service provider application 102b, the healthcare service providers provide good faith estimates for healthcare services costs including combinations of particular procedures associated with each healthcare service.

The software patient applications 102a, 102b are configured to communicate with a server system 110 via a communication network 108. The server system 110 provides the functionalities offered by the system 100 and the applications 102a, 102b provide compliance with healthcare regulations.

The server system 110 includes a patient database 112 which is configured to store patient profiles, including login credentials, service preferences, and past healthcare service quote requests. The database 112 is adapted to be updated in real-time and includes the information of all registered patients of the system 100. A healthcare service provider database 114 stores information of the registered healthcare service providers, corresponding offered healthcare services, pricing details, and automated response configurations for frequently requested services.

An automated quote module 116 generates good faith estimates based on provider data and patient requests. The module 116 is configured to process patient requests and generates estimates based on predefined rules and provider inputs. The generated estimates can be shown to both the providers and the patients, as well as providing for a benchmark for the estimates. An application server 118 hosts the back-end application logic, processes patient requests, and manages the system functionality. The application server 118 processes and manages the database operations of the databases 112, 114. A payment module 120 enables patients to make secure payments for selected services directly through the system 100. The payment module 120 is adapted to deduct a commission percentage from the payment and credits the payment to the healthcare service providers. The payment module 120 may use third party payment gateways for facilitating the payments between the patients and the providers.

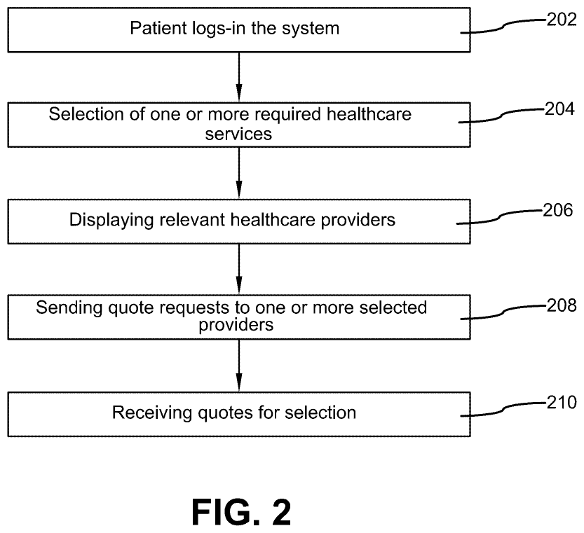

FIG. 2 illustrates a flow chart depicting a process of providing healthcare service quotes to patients in accordance with one embodiment of the present invention. Initially, a patient logs-in the system 100 using the patient application 102a (Step 202). A patient can login using credentials such as user id, password and may access the system 100 upon successful authorization of the credentials. Then, the patient may select one or more required/needed healthcare services for which the estimates are required (Step 204). In some embodiments, the patient may upload their medical reports and the application 102a may recommend a few healthcare services names. Based on the received healthcare services information, the application 102a displays a list of relevant healthcare services providers (Step 206). The patient may provide additional criteria such as location, maximum budget, and more.

Thereafter, the patient selects one or more service providers from the list and sends healthcare services quote requests from the selected providers (Step 208). In some embodiments, the patient can blacklist a provider which the healthcare service estimation platform 100 would not recommend for the future requests. Finally, the quotes are received from the selected providers and are displayed by the application for review and selection by the patient (Step 210). In some embodiments, the estimate generated by the module 116 may also be displayed along with the quotes of the service providers. It is to be appreciated that the estimate can include a listing and separate estimate for each procedure associated with the healthcare service.

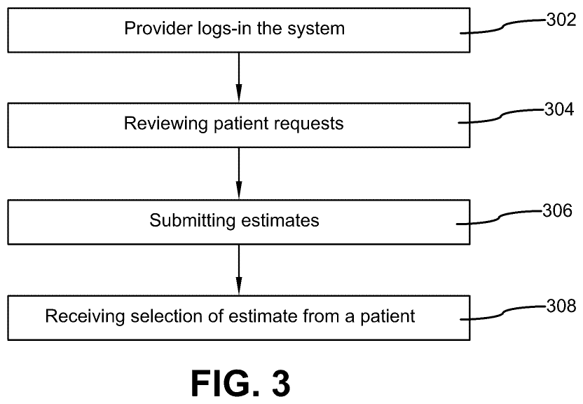

FIG. 3 illustrates a flow chart depicting a provider-side process for providing estimates of healthcare services in accordance with one embodiment of the present invention. Initially, a service provider logs-in the system 100 using the provider application 102b (Step 302). The service provider can register to use the services offered by the healthcare service estimation platform 100 and may login by using the credentials which are authenticated by the service provider application 102b. Then, the healthcare service provider can review the patient requests received for estimates of healthcare services (Step 304). It should be noted that one patient request is sent to a plurality of providers for estimates wherein the providers are automatically selected based on criteria of the patient. Based on preference of the provider, the provider may provide estimates to one or more of the received estimate requests (Step 306). The provider database 114 maintains a list of estimates provided by the providers and the healthcare cost transparency system 100 may delist one or more providers on providing an inflated estimate. Finally, the provider receives a selection of their estimate by a patient and when, estimate is not selected by the patient, a notification is provided to the provider (Step 308).

In the preferred embodiment, healthcare service providers can configure pre-configured responses for frequently requested services. The response can be automatically sent to a patient when the provider receives a corresponding frequent service request.

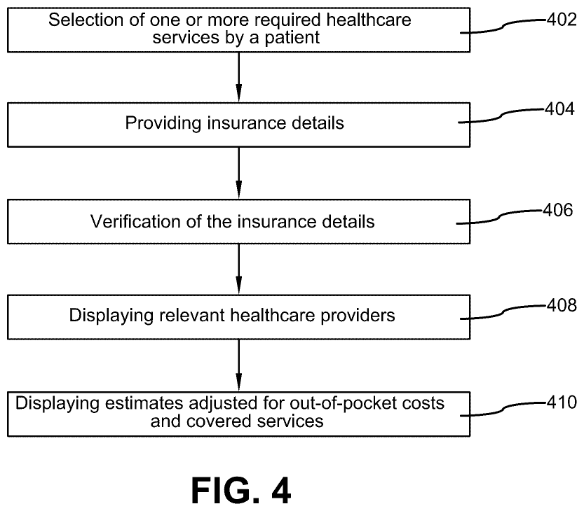

FIG. 4 illustrates a flow chart depicting a process of generating estimates for healthcare services considering insurance of the patients in accordance with the disclosed architecture. Initially, one or more providers are selected by a user for providing a service such as described in FIG. 2 (Step 402). Then, the insurance details are provided by the patient which may include the insurance company, insurance policy number (Step 404). The steps 402, 404 can be interchanged depending on the requirements of the users and design of the system 100. Thereafter, verification is done of the insurance policy which enables the system to provide an adjusted estimate for the service request considering the health insurance policy (Step 406). Based on the insurance policy and the patient's request, relevant healthcare service providers are displayed to the patient by the patient application 102a (Step 408). Finally, out-of-pocket costs and covered services are displayed for the selected service providers (Step 410).

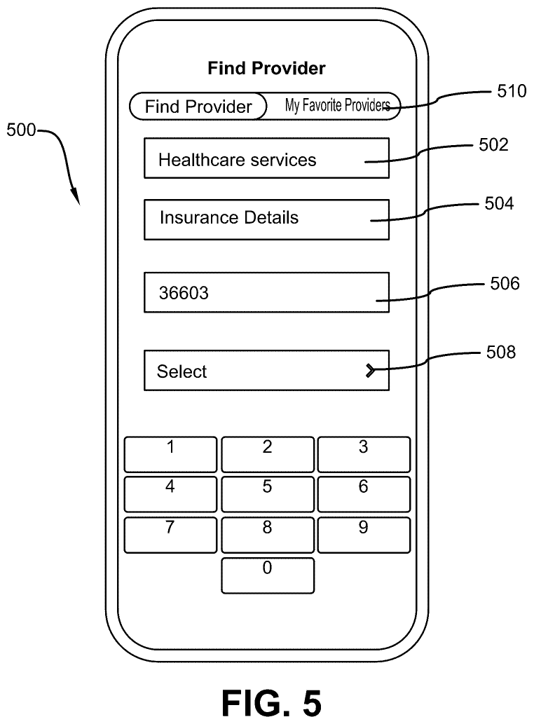

FIG. 5 illustrates an exemplary user interface displayed by the patient application for requesting service providers in accordance with one embodiment of the present invention. As illustrated, the interface 500 provides a healthcare services search box 502 for inputting or selecting at least one healthcare service request. An option insurance details box 504 enables patients to input insurance details which can be used in generating an estimate for the patient. Other request criteria such as location for service 506 and distance from the location 508 can also be provided by the patient. The interface 500 provides a ‘favorite’ providers tab 510 which may show the service providers marked as favorite by the patient.

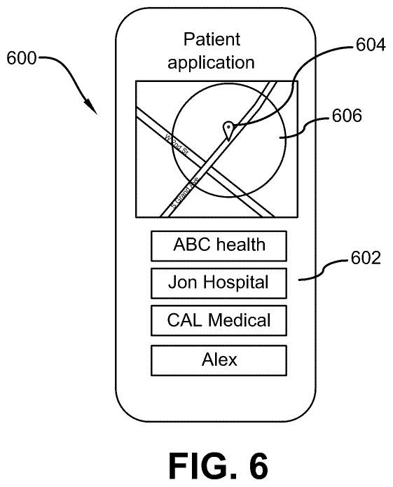

FIG. 6 illustrates another user interface displayed by the patient application showing a list of relevant healthcare services providers in accordance with the disclosed architecture. Based on matching of patient's criteria such as location, urgency, and service type and service provider's specialty and location, a list 602 of plurality of providers is displayed on the interface 600. In some embodiments, location of service 604 and a geographic range 606 preset by the patient is also displayed in the interface 600. It should be noted that favorite providers and highest ranked providers may be displayed in different colors or different identifiers.

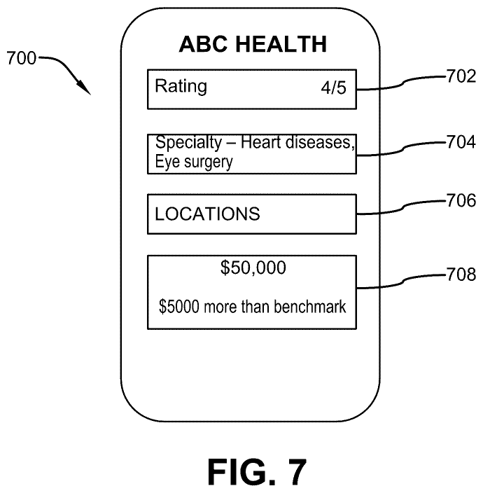

FIG. 7 illustrates an exemplary user interface for displaying information of a provider which enables a patient to make an informed decision in accordance with the disclosed architecture. Any service provider displayed in the list 602 can be selected by the patient to display information about the provider such as shown in FIG. 7. As illustrated, rating 702 of the provider is displayed which is based on the ratings provided by patients of the system. Specialty information 704 of the provider is also displayed by the interface 700 which enables the patient to make an informed decision on selection of the provider. Different locations 706 in which the provider is available is also displayed in the interface 700 and any location can be selected by the patient for using the requested healthcare services. The quote or estimate 708, including estimates of each procedure associated with the healthcare service, provided by provider along with the comparison with the automatic generated benchmark is displayed in the interface 700 and the provider can be selected by the patient for availing the services.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “healthcare service estimating system”, “healthcare service estimation platform”, “healthcare cost transparency system”, and “system” are interchangeable and refer to the system and method for healthcare service estimating system 100 of the present invention.

Notwithstanding the forgoing, the system and method for healthcare service estimating system 100 of the present invention can be of any suitable size and configuration as is known in the art without affecting the overall concept of the invention, provided that it accomplishes the above stated objectives. One of ordinary skill in the art will appreciate that the system and method for healthcare service estimating system 100 as shown in the FIGS. are for illustrative purposes only, and that many other configurations of the system and method for healthcare service estimating system 100 are well within the scope of the present disclosure.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A healthcare service estimating system comprising:
a plurality of healthcare services' costs;
a listing of healthcare service providers;
a patient application;
a healthcare service provider application;
a patient;
an electronic computing device;
a provider device; and
a communication network;
wherein said plurality of healthcare services' costs are estimates for healthcare services;
wherein said patient application is installed on said electronic computing device;
wherein said patient receives said plurality of healthcare services' costs from said listing of healthcare service providers through said patient application;
wherein said patient application provides said patient a user interface for enabling a user to input healthcare services requirements and receive said plurality of healthcare services' costs from said listing of healthcare service providers;
wherein said healthcare service provider application installed in said provider device; and
further wherein said healthcare service provider application having functionalities selected from the group consisting of manage profiles, view patient requests, and respond to quote inquiries of said patient for said estimates for healthcare services.

2. The healthcare service estimating system of claim 1 further comprising a server, wherein said server communicates with said patient application and said healthcare service provider application via said communication network.

3. The healthcare service estimating system of claim 2, wherein said server having a patient database including information selected from the group consisting of a patient profile, a login credential, a service preference, and a past said quote inquiry of an offered healthcare service.

4. The healthcare service estimating system of claim 3, wherein said server having a healthcare service provider database including information selected from the group consisting of said listing of healthcare service providers, said offered healthcare services, said estimates for healthcare services, and automated response configurations for frequently requested services.

5. The healthcare service estimating system of claim 4, wherein said electronic computing device is a smartphone.

6. The healthcare service estimating system of claim 4 further comprising an automated quote module generating said estimates for healthcare services.

7. The healthcare service estimating system of claim 6, wherein said estimates for healthcare services are based on information selected from the group consisting of a provider data, a patient request, a predefined rule, a provider input, and a combination of multiple procedures associated with said estimates for healthcare services.

8. A healthcare service estimating system comprising:
a plurality of healthcare services' costs;
a listing of healthcare service providers;
a patient application;
a healthcare service provider application;
a patient;
an electronic computing device;
a provider device;
a communication network; and
a payment module;
wherein said plurality of healthcare services' costs are estimates for healthcare services;
wherein said patient application is installed on said electronic computing device;
wherein said patient receives said plurality of healthcare services' costs from said listing of healthcare service providers through said patient application;
wherein said patient application provides said patient a user interface for enabling a user to input healthcare services requirements and receive said plurality of healthcare services' costs from said listing of healthcare service providers;
wherein said healthcare service provider application installed in said provider device;
wherein said healthcare service provider application having functionalities selected from the group consisting of manage profiles, view patient requests, and respond to quote inquiries of said patient for said estimates for healthcare services;
wherein said patient uses said payment module to make a payment for selected said healthcare services; and
further wherein said payment module deducts a commission percentage from said payment and credits said payment to said listing of healthcare service providers.

9. The healthcare service estimating system of claim 8, wherein said payment module having a third party payment gateway for processing said payment between said patient and said listing of healthcare service providers.

10. The healthcare service estimating system of claim 9 further comprising a server, wherein said server communicates with said patient application and said healthcare service provider application via said communication network.

11. The healthcare service estimating system of claim 10, wherein said server having a patient database including information selected from the group consisting of a patient profile, a login credential, a service preference, and a past said quote inquiry of an offered healthcare service.

12. The healthcare service estimating system of claim 11, wherein said server having a healthcare service provider database including information selected from the group consisting of said listing of healthcare service providers, said offered healthcare services, said estimates for healthcare services, and automated response configurations for frequently requested services.

13. The healthcare service estimating system of claim 12, wherein said electronic computing device is a smartphone.

14. The healthcare service estimating system of claim 12 further comprising an automated quote module generating said estimates for healthcare services.

15. The healthcare service estimating system of claim 14, wherein said estimates for healthcare services are based on information selected from the group consisting of a provider data, a patient request, a predefined rule, a provider input, and a combination of multiple procedures associated with said estimates for healthcare services.

16. A method of comparing healthcare service costs, the method comprising the steps of:
providing a plurality of healthcare services' costs; a listing of healthcare service providers; a patient application; a healthcare service provider application; a patient; an electronic computing device; a provider device; a communication network; and a payment module, wherein said plurality of healthcare services' costs are estimates for healthcare services;
installing said patient application on said electronic computing device;
receiving said plurality of healthcare services' costs from said listing of healthcare service providers through said patient application;
providing said patient with said patient application a user interface for enabling a user to input healthcare services requirements and receive said plurality of healthcare services' costs from said listing of healthcare service providers; and
installing said healthcare service provider application in said provider device, wherein said healthcare service provider application having functionalities selected from the group consisting of manage profiles, view patient requests, and respond to quote inquiries of said patient for said estimates for healthcare services.

17. The method of comparing healthcare service costs of claim 16, further comprising a step of displaying said listing of healthcare services providers on said patient application based on said healthcare services requirements.

18. The method of comparing healthcare service costs of claim 17, further comprising the steps of receiving said estimates for healthcare services from one or more of said listing of healthcare services providers, displaying said estimates for healthcare services on said patent application, and selecting by said patient one or more of said listing of healthcare services providers.

19. The method of comparing healthcare service costs of claim 17, further comprising the steps of uploading a medical report to said patient application and recommending said listing of healthcare services providers based on said medical report.

20. The method of comparing healthcare service costs of claim 16, further comprising a step of using said payment module by said patient to make a payment for selected said healthcare services, wherein said payment module deducts a commission percentage from said payment and credits said payment to said listing of healthcare service providers.
